(19) United States
(12) Patent Application Publication
(10) Pub. No.: US 2004/0001789 A1
(43) Pub. Date: Jan. 1, 2004

(54) Cytotoxicity mediation of cells evidencing surface expression of gp96 or precursors thereof

(76) Inventors: David S. F. Young, Toronto (CA); Miyoko Takahashi, North York (CA); Susan E. Hahn, Toronto (CA)

Correspondence Address: Michael A. Slavin, McHale & Slavin, P.A., Suite 402, 4440 PGA Boulevard, Palm Beach Gardens, FL, 33410, US

(21) Appl. No.: 10/403,516

(22) Filed: Mar. 31, 2003

Related U.S. Application Data
(63) Continuation-in-part of application No. 09/727,361, filed on Nov. 29, 2000.
Application No. 09/727,361 is a continuation-in-part of application No. 09/415,278, filed on Oct. 8, 1999, now Pat. No. 6,180,357.

(51) Int. Cl. A61K 51/00; A61K 39/395

(52) U.S. Cl. 424/1.49; 424/155.1; 424/132.1; 424/178.1

(57) ABSTRACT

This invention relates to the diagnosis and treatment of cancerous diseases, particularly to the mediation of cytotoxicity of tumor cells; and most particularly to the use of cancerous disease modifying antibodies (CDMAB), optionally in combination with one or more chemotherapeutic agents, as a means for initiating the cytotoxic response. The invention further relates to binding assays which utilize the CDMABs of the instant invention.

### REFERENCE TO RELATED APPLICATIONS

This application is a continuation-in-part of application Ser. No. 09/727,361, filed Nov. 29, 2000, which is a continuation-in-part of application Ser. No. 09/415,278, filed Oct. 8, 1999, now U.S. Pat. No. 6,180,357 B1, the contents of each of which are herein incorporated by reference.

### FIELD OF THE INVENTION

This invention relates to the diagnosis and treatment of cancerous diseases, particularly to the mediation of cytotoxicity of tumor cells; and most particularly to the use of cancerous disease modifying antibodies (CDMAB), optionally in combination with one or more chemotherapeutic agents, as a means for initiating the cytotoxic response. The invention further relates to binding assays which utilize the CDMABs of the instant invention.

### BACKGROUND OF THE INVENTION

The immunogenicity of tumors has been long observed, and the identification of as a molecule involved with tumor-specific rejection suggests a function for this stress protein beyond its role as a molecular chaperone. Srivastava and his colleagues have previously shown chemically induced murine sarcomas were immunogenic in synegic hosts and previous exposure was protective from subsequent tumor transplants. The protection was specific to the tumor and did not offer cross-protection from other syngenic sarcomas. A key observation was that preparations of gp96 purified from a tumor conferred protection for that tumor, but not for antigenically distinct tumors. Depletion of gp96 abrogated the protection, and although gp96 was also found in normal tissues, gp96 prepared from normal tissue did not confer protection in those studies. Sequencing of gp96 from different sources, including tumors, showed sequence identity and did not contain distinct tumor associated antigen sequences. Observations that gp96 was highly homologous to molecular chaperone hsp90 and possibly identical to glucose regulated protein grp94 suggested that gp96 may, through protein chaperoning, bind tumor antigens. The tumor specific protection offered by gp96 may not be due to gp96 itself but may be due to the antigenic peptides carried by gp96. Additional evidence for the role of gp96 in chaperoning immunogenic peptides include induction of beta-galactosidase specific cytotoxic T-lymphocytes (CTL) by beta-galactosidase overexpressing cell lines, and the induction of VSV specific CTL by VSV infected cells mediated through immunization with associated purified gp96.

Further studies showed that gp96 immunization primed CD8 cells. This required professional antigen presenting cells, specifically macrophages, but not CD4 cells. However, both CD4 and CD8 cells were necessary for the effector phase of the anti-tumor response in addition to macrophages. These studies demonstrated macrophage-dependent and independent paths of tumor immunity and suggested the possible role of gp96 in the transfer of immunogenic peptides to macrophage MHC class 1 molecules. The identification of CD91 as a receptor for gp96 is consistent with its role in T-cell activation. CD91 has previously been described as the receptor for alpha2-macroglobulin and is related to the low density lipoprotein receptor. This receptor is present in monocytes, hepatocytes, fibroblasts, and keratinocytes and has been shown to participate in T-cell activation. Peptides chaperoned by gp96 can be re-presented by MHC class 1 molecules through engagement of CD91 and can induce release of cytokines such as IFN-gamma. This would appear to suggest another arm of adaptive immunity imparted by gp96.

Gp96 is a member of the heat shock protein (hsp) family. Heat shock proteins were originally identified in Drosophila in 1962, and the glucose regulated members of this family were identified in 1977, and then subsequently characterized. The first monoclonal antibody directed against the 100 kD heat stress glycoprotein, corresponding to gp96, was produced in 1983.

In accordance with the instant specification, the term gp96 will be understood to include both the gp96 protein itself and gp96 precursor protein.

Comparison of human to murine gp96 showed 96% identity and there was also significant similarity to stress proteins such as the 94 kD glucose-regulated protein (grp94), the 108 kD heat shock protein (hsp 108) and the 99 kD endoplasmic reticular protein (ERp99). The gene for human gp96 ((TRA1, mapped to chromosome 12, 12 (q24.2→q24.3)), did not show restriction fragment polymorphism among different individuals and did not have splice variants. This was in contrast to hsp90, the cytosolic paralog of gp96, which has two encoding genes that produce two isoforms (alpha and beta). Despite being encoded by a single gene, gp96 can be heterogeneous on an SDS-PAGE gel. As many as three bands can be observed, with migration between 96 kD and 110 kD, which may be due to phosphorylation, conformational changes, and less likely, glycosylation. Gp96 has been shown to be phylogenically conserved across Metazoa, appearing on cell surfaces in species as diverse as xenopus, hagfish, channel catfish, as well as mammalian cells. Not only has the gene been relatively conserved, the immunological function has also been conserved as shown by the ability of xenopus gp96 to chaperone peptides for presentation by murine antigen presenting cells to murine CD8 cells.

The gp96 protein, in its native state, may exist as obligate tail-to-tail tri-nodular homodimers, which autonomously dimerize non-covalently through a 20 kD C-terminal domain corresponding to a 44 amino acid hydrophobic region. As the most abundant lumenal ER protein, gp96 is synthesized with an N-terminal signal sequence, and a C-terminal KDEL ER retention sequence. Gp96 is glycosylated with a single high mannose oligosaccharide at Asn196. The biochemical function of gp96 is not well understood, but it is thought to act as a molecular chaperone within the ER and there has been evidence that gp96 assists in the folding and export of soluble and membrane-bound oligomeric proteins, including immunoglobulins, epidermal growth factor receptor, Toll-like receptors 1, 2, and 4, integrins CD11a, CD18, and CD49d. The protein can be induced by glucose starvation, calcium ionophores, malfolded proteins in the ER, etc. A peptide binding pocket has tentatively been mapped to amino acid residues 624-630 which abuts the dimerization domain. In support of the peptide binding role of gp96, peptides with sequence similarity to HBV core protein have been extracted from gp96 derived from HBV induced hepatocellular caricinomas. Consistent with its chaperone role, there has been evidence of kinase activity localized to the C-terminal end that may be involved in the loading of protein onto gp96. An ATP-binding cassette has been described, and Mg(2+)-dependent ATPase activity has been demonstrated.

Although gp96 is primarily an ER resident molecule, there has been evidence for its cell surface expression in cancers as diverse as Xenopus lymphoid tumors to murine sarcoma cells. In Meth A (Mouse) fibrosarcoma cells, cell surface gp96 represented 2-3% of the total estimated gp96. The cell surface location of gp96 is likely not due to adventitious uptake of released gp96 as it is inhibited by Brefeldin A, and there is an associated increase in cell surface expression in response to heat shock or reducing agents. The cell surface bound gp96 still retains the KDEL sequence, and requires four hours for re-expression after pronase treatment which suggests a protein trafficking cycle.

Several possibilities have been proposed to explain gp96 cell surface expression such as severe metabolic alterations leading to disturbances in the secretory pathways or differential expression of HSP anchoring proteins in transformed/infected versus normal cells. It may also be that gp96 is carried to the cell surface by the proteins it chaperones as is the case with hsp70/transferrin or hsp90/MHC-I or beta2-microglobulin in which pairs of proteins co-localize to the cell surface.

Despite the predominance of evidence that suggests cell surface expression of gp96 is pathological, there has been some contrary evidence for normal physiological function. In one instance three membrane antigens of 90 kD, 110 kD, and 180 kD were identified by HDL ligand blotting in normal cells. These antigens were purified followed by N-terminal sequencing, and shown to have high homology to human gp96. However, immunogold EM studies with antibodies made against the isolated proteins did not label the ER, which is inconsistent with gp96 distribution, whereas antibodies made to human gp96 did show an ER distribution of the antigen. Attempts to simulate cell surface expression with KDEL deficient mutants produced expression in only a small percentage of cells. This suggests that cell surface expression is not due to escape by KDEL deletion, despite some evidence that chicken oviduct, rat or chicken liver purified gp96 do not have intact KDEL sequences. Further, HDL uptake by these transfectants were at best less than two-fold greater than control transfectants. These experiments would appear to suggest that gp96 is an HDL receptor because of the HDL ligand blotting. In another example of homology, a previously described cell surface glycoprotein, the B2, B-lymphocyte co-stimulatory molecule was shown to be essentially identical to gp96. Another group has also identified gp96 on cell surfaces of IgM+B-cell splenocytes. This, in combination with the known role of CD91 in T-cell activation and its identification as the receptor for gp96 brings together another pair of co-stimulatory molecules. Thus, although there is evidence for a physiological role for cell surface gp96, the preponderance of evidence is that its role is immunologic and related to pathological processes

In addition to the substantial evidence for gp96 mediated tumor rejection in animal models, there have been two lines of inquiry that enjoin that research with human relevance. In the first case, many groups have shown the over-expression of gp96 in human cancers. For example, by immunochemistry, gp96 was shown to be overexpressed in well-differentiated esophageal adenocarcinoma and gastric adenocarcinoma, but not in normal esophagus or esophageal squamous cell carcinoma. In another study there was increased tumor-to-stroma gp96 expression in metastatic primary colorectal carcinoma (CRC; 76%) compared to non-metastatic CRC (45%). In hepatocellular carcinoma (HCC), by both Northern analysis and immunohistochemistry, there was increased expression of gp96 in HCC compared to normal liver and especially in moderately-to-poorly differentiated and in poorly differentiated HCC. In breast cancer, in a series of 8 patients, there has been evidence of concomitant increase in gp96 expression with IL-6 expression, and, IL-6 has been shown to induce gp96 in vitro. In lung squamous cell carcinoma, using a suppression subtractive hybridization approach, gp96 has been shown to be upregulated.

A second line of inquiry that establishes the clinical relevance of gp96 has been the initial clinical trials carried out with gp96. In a pilot study 16 patients with assorted advanced cancers were administered gp96 protein preparations derived from their tumors four times weekly at a dose of 25 micrograms s.c. There were six clinical responders, with three still alive at the time of the report (approx 4 yrs from enrollment of the last patient), with about half the patients showing an immunological response. In a melanoma trial 64 patients were enrolled for vaccination with gp96 preparations from their tumors. There were 39 evaluable patients with the remainder dropping out for death, disease progression, treatment with chemotherapy or inability to produce enough vaccine. There were 28 patients with measurable tumors and 11 patients without. 23 of the 28 patients were evaluated for immunological response and 11 patients showed some immunological response. Of the 28 tumor-bearing patients, there were 8 clinical responders of whom there were 2 complete responders and three with stable disease. 12 of the 28 patients did not respond to therapy. These two studies show that gp96 is relevant in the clinic and suggest a gp96-based therapy can have impact on cancer.

From both the pre-clinical and clinical research there seems to be a role for gp96 in the therapy of cancer. Since gp96 is expressed preferentially on the cell surface of cancer cells, it would not be unusual to raise an antibody to this protein when cancer cells are used as an immunogen. What is surprising is that the instantly disclosed H460-16-2 cancerous disease modifying antibody, upon binding with the gp96 or gp96 precursor molecule receptors, can effectively induce cancer specific cell death in-vitro and control cancer in vivo. Although the precise mechanism needs to be clarified, the essential elements for antibody induced cytotoxicity in cancer, and its in vivo effects, are satisfied by the biochemical and biological properties of gp96.

The evidence includes the facts that there is substantial evidence for the expression of gp96 on the cell surface, that expression is associated with cellular perturbations such as oncologic transformation or viral infection (examples of cellular stress), that there is a differential expression of gp96 between cancer and normal cells, that there is an increased expression of the glycoprotein in cancer cells (as seen in human tissue surveys), that cancer cells are susceptible to gp96 induced immunity, both in the laboratory, and in the clinic, that tumors can be controlled by associated gp96 immunity in vivo, that specific cytotoxicity is conferred by tumor derived gp96, and that bystander cells and tissues are relatively unharmed.

What is not obvious is how an antibody can induce cytotoxicity via gp96 since, to date, there has not been a signaling role for this glycoprotein. The suggestion that gp96 may be transported to the cell surface as a byproduct of its chaperoning function also brings up the possibility that this association is maintained once at the cell surface. This is consistent with the evidence that gp96 binds tightly, almost irreversibly in some cases, to its chaperoned peptides. To the extent that gp96 is associated with signaling proteins on the cell surface, a gp96 antibody may induce clustering of these proteins through oligimerization of gp96 via divalent antibodies, such as IgGs. It is likely that the ability of a gp96 antibody to induce cell death is highly dependent on the antigenic epitope recognized by that antibody, which may be a very specific conformer that may or may not be modified by a cancer specific antigen.

Thus, if an antibody composition were isolated which mediated cancerous cell cytotoxicity, as a function of its attraction to cell surface expression of gp96 on said cells, a valuable diagnostic and therapeutic procedure would be realized.

Prior Patents:

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas and not dependent upon the epithelial tissue of origin.

U.S. Pat. No. 5,783,186 is drawn to Anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an antinuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal antinuclear autoantibody from an aged mammal, and a hybridoma cell line producing a monoclonal antinuclear autoantibody.

### SUMMARY OF THE INVENTION

The instant inventors have previously been awarded U.S. Pat. No. 6,180,357, entitled “Individualized Patient Specific Anti-Cancer Antibodies” the entire contents of which are herein incorporated by reference, directed to a process for selecting individually customized anti-cancer antibodies which are useful in treating a cancerous disease.

This application utilizes the method for producing patient specific anti-cancer antibodies as taught in the '357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases.

Accordingly, it is an objective of the invention to provide a method for utilizing cancerous disease modifying antibodies from cells derived from a particular individual which are cytotoxic with respect to cancer cells while simultaneously being relatively non-toxic to non-cancerous cells, in order to mediate cell death of tumor cells.

A still further objective of the instant invention is to utilize cancerous disease modifying antibodies in a diagnostic assay for diagnosis, prognosis, and monitoring of a cancerous condition.

Other objects and advantages of this invention will become apparent from the following description wherein are set forth, by way of illustration and example, certain embodiments of this invention.

### BRIEF DESCRIPTION OF THE FIGURES

FIG. 1. Western blot of MDA-MB-468 membranes probed with H460-16-2. Lane 1: Membrane proteins separated under reducing conditions. Lane 2: Membrane proteins separated under non-reducing conditions. Molecular weight markers are indicated on the left;

FIG. 2. 2-Dimensional Western blot and SDS-PAGE gel of MDA-MB-468 membrane proteins. Panel A demonstrates the position of the 2 proteins recognized by H460-16-2. Panel B demonstrates a similar blot probed with an isotype control antibody. Panel C shows a SYPRO Ruby-stained gel of MDA-MB-468 membranes. Arrows indicate the position of protein spots corresponding to panel A;

FIG. 3. Effect of deglycosylation on the binding of H460-16-2 to MDA-MB-231 membranes. Panel A demonstrates the binding of H460-16-2 in a Western blot to untreated MDA-MB-231 cell membranes (Lane 2), membranes treated with glycosidases (see text) at 37° C. for 24 hr (Lane 3), and membranes treated with glycosidases at 25° C. for 24 hr (Lane 4). Lane 1 shows the position of the molecular weight markers. Panel B demonstrates the binding of the high-mannose binding lectin GNA to a similar blot;

FIG. 4. Western blot using anti-gp96 antibody. Western blot demonstrated the binding of anti-gp96 (clone 9G10.F8.2) to membranes from MDA-MB-468 (Lane 1), MDA-MB-231 (Lane 2) and proteins immunoprecipitated with H460-16-2 antibody from MDA-MB-468 membranes (Lane 3);

FIG. 5. Representative histograms for H460-16-2 binding by FACS. Histograms for binding of H460-16-2 (20 μg/mL), 107.3 isotype control (20 μ/mL) and anti EGFR (5 μg/mL) are presented for breast cancer (MDA-MB-231 and MDA-MB-468) and normal (Hs578.Bst and CCD27sk) cell lines.

### DETAILED DESCRIPTION OF THE INVENTION

### EXAMPLE 1

### Identification of Binding Proteins by Western Blotting

To identify the antigen(s) recognized by the antibody H460-16-2, cell membranes expressing this antigen were subjected to gel electrophoresis, and transferred to membranes. Western blotting was used to determine proteins detected by this antibody. Spots on a parallel stained gel corresponding to the Western blot were excised, and identified using mass spectroscopy.

1. Membrane Preparation

Previous work demonstrated binding by FACS of H460-16-2 to the breast cancer cell lines MDA-MB-231 (MB-231) and MDA-MB-468 (MB-468). Accordingly, membrane preparations from these two cell lines were used for antigen identification. Total cell membranes were prepared from confluent cultures of MB-231 or MB-468 breast cancer cells. Media was removed from flasks, and the cells were washed three times with PBS. After the final wash, cells were dissociated with Dissociation Buffer (Gibco-BRL; Grand Island, N.Y.) for 5 min. at 37° C. Cells were collected and centrifuged at 1200 rpm for 10 minutes at 4° C. After centrifugation, cell pellets were resuspended in 1 mL of hypotonic lysis buffer containing 10 micrograms/mL leupeptin, 10 micrograms/mL aprotonin and 25 micrograms/mL 4-(2-aminoethyl)-benzenesulfonyl fluoride. Cells were then lysed using 5 cycles of rapid freezing and thawing. Cell lysates were centrifuged at 9500 rpm for 10 min. at 4° C. to remove nuclear particulate. Supernatant was harvested, and then centrifuged at 75,000×g for 57 min., at 4° C. Supernatant was carefully removed from tubes, and pellets were resuspended in 0.5 to 1 mL of hypotonic lysis buffer containing 1% Triton X-100. Membranes were then assayed for protein content, and stored at 80° C.

2. 1-Dimensional SDS-PAGE

Membrane proteins were separated by 1-dimensional SDS-polyacrylamide gel electrophoresis. 20 micrograms of membrane protein was loaded onto a lane of a 12% SDS-PAGE gel. A sample of pre-stained molecular weight markers (Biorad; Mississauga, ON) was loaded in a reference lane. The sample was separated by electrophoresis under non-reducing conditions, in the absence of dithiothreitol (DTT). Electrophoresis was carried out at 100 V for 10 min., followed by 65 min. at 150 V. Proteins were transferred from the gel to PVDF (Millipore; Billerica, Mass.) membranes by electroblotting for 16 hr at 40 V. Quantitative transfer was assessed by noting complete transfer of the prestained markers from the gel to the membrane.

After transfer, membranes were blocked with 5% skim milk powder in Tris-buffered saline containing 0.5% Tween (TBST) for 2 hr. Membranes were then incubated with 2-2.5 micrograms/mL H460-16-2 diluted into 3% skim milk powder in TBST for 2 hr. After washing 3 times with TBST, membranes were incubated with goat anti-mouse IgG (Fc) conjugated to horse radish peroxidase (HRP) from Jackson Immunologicals (West Grove, Pa.) for 1 hr. This incubation was followed by washing 3 times with TBST, followed by incubation with the HRP substrate TMB (substrate kit from Vector Laboratories; Burlington, ON).

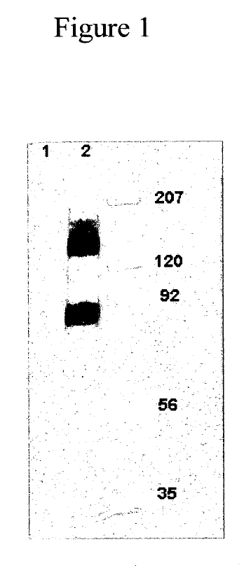

FIG. 1 demonstrates the results of the Western blotting as described. H460-16-2 binds clearly to 2 molecular weight (MW) regions of the separated MB-468 membrane proteins. By comparison to the molecular weight standards, the antibody binds to proteins of MW 85-95 kD and MW 130-150 kD. The epitope recognized by the antibody H460-16-2 appears to be to a conformational epitope, since the antibody was unable to bind spots transferred from gels under reducing conditions in the presence of DTT.

3. 2-Dimensional SDS-PAGE

In order to obtain better resolution of the binding entities, and to facilitate extraction and identification of the proteins, 2-dimensional electrophoresis was carried out. Total membrane proteins (75-200 micrograms) prepared as described were precipitated using the PlusOne 2-D Clean Up kit (Amersham; Baie D'Urfé, QC)), and then resuspended in rehydration buffer containing ampholytes in the pH range 3-10. Samples were centrifuged to remove particulate material, and then loaded onto IPG strips (Amersham) in the presence of a rehydration solution. Proteins were focused using the following protocol: 16 hr for rehydration; 500 V, 250 Vhrs, 1000 V, 500 Vhrs; 5000 V, 7500 V hrs. The strip was then removed from the strip holders, and incubated in an SDS-PAGE equilibration buffer. After 15 min., the strip was placed on the top of an 8% gel, and sealed with an agarose solution. Prestained MW markers were loaded beside the strip. Electrophoresis was carried out at 100 V for 10 min., followed by 65 min. at 150 V. One of the gels was fixed for 30 min. with 10% methanol/7% acetic acid, and then stained with the fluorescent dye SYPRO Ruby™ (Molecular Probes, Eugene, Oreg.). Protein spots were visualized under UV light. From a second and third gel, proteins were transferred from the gels to PVDF (Millipore) membranes by electroblotting for 16 hr at 40 V. Quantitative transfer was assessed by noting complete transfer of the prestained markers from the gel to the membrane.

After transfer, membranes were blocked with 5% skim milk powder in Tris-buffered saline containing 0.5% Tween (TBST) for 2 hr. One of the membranes was then incubated with 2-2.5 micrograms/mL H460-16-2 diluted into 3% skim milk powder in TBST for 2 hrs. A similar membrane was incubated with the same concentration of an isotype control (Mouse anti-trinitrophenol, IgG1,k; clone 107.3 (BD Biosciences, Oakville, ON)). After washing 3 times with TBST, membranes were incubated with goat anti-mouse IgG (Fc) conjugated to horse radish peroxidase (HRP) from Jackson Laboratories for 1 hr. This incubation was followed by washing 3 times with TBST, followed by incubation with the HRP substrate TMB (substrate kit from Vector Laboratories).

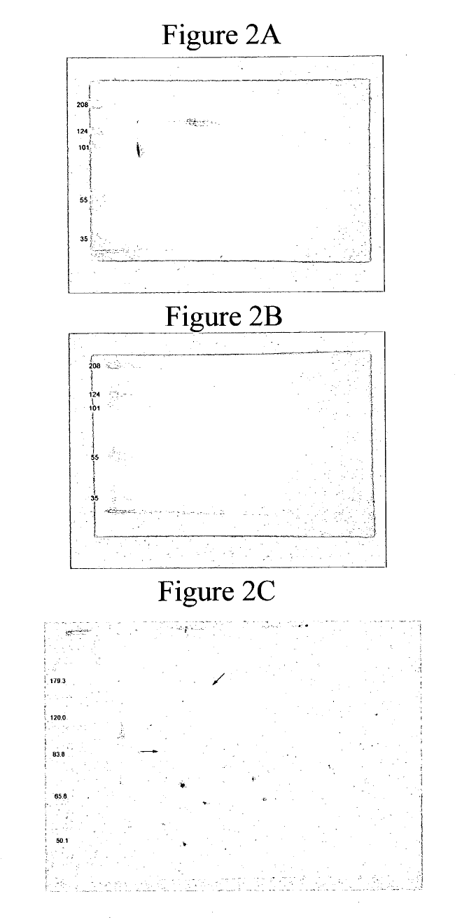

FIG. 2a demonstrates the Western blot obtained from membranes incubated with H460-16-2. Two distinct binding spots are observed, with molecular weights corresponding with those obtained by 1-dimensional electrophoresis. One is observed at a MW of approximately 85-95 kD according to the MW standards, and is in the acidic portion of the gel with an estimated pI of 3-4. The second spot is in the MW range of 130-150 kD according to the MW standards, and has a pI more basic than the 85-95 kD protein.

FIG. 2b demonstrates the Western blot obtained from membranes incubated with the isotype control antibody. No spots were visible on this blot, indicating that the binding of H460-16-2 was not due to non-specific binding.

FIG. 2c shows a SYPRO Ruby™ stained 2D-gel of MB-468 membrane proteins.

### EXAMPLE 2

### Identification of Antigens Bound by H460-16-2

Using the approach in Example 1, the regions of the gel corresponding to the spots recognized by H460-16-2 on Western blots were excised using a sterile pipette tip. These gel plugs were then used for identification of proteins by mass spectroscopy.

The samples were subjected to robotic in-gel digestion using trypsin (ProGest) and a portion of the resulting digest supernatant was used for MALDI/MS analysis. Spotting was performed robotically (ProMS) with ZipTips; peptides were eluted from the C18 material with matrix (α-cyano 4-hydroxy cinnamic acid) prepared in 60% acetonitrile, 0.2% TFA. MALDI/MS data was acquired on an Applied Biosystems Voyager DE-STR instrument and the observed m/z values were submitted to ProFound (Proteometrics software package) for peptide mass fingerprint searching. ProFound queried a locally stored copy of the NCBInr database. Those samples that proved inconclusive following MALDI/MS were analyzed by nano LC/MS/MS on a Micromass Q-Tof2 using a 75 μm C18 column at a flow-rate of 200 nL/min. The MS/MS data were searched using a local copy of MASCOT.

Two independent experiments were carried out, using MB-468 and MB-231 membranes, respectively. In both membrane preparations, a distinct 85-95 kD protein is observed by Western blotting, but this protein is proportionately more intense in MB-231 membranes. The protein observed at approximately 130-150 kD is more prevalent in MB-468 cell membranes. The proteins obtained from MALDI and LC/MS/MS analysis of these two experiments are listed in Tables 1 and 2. For those proteins identified by LC/MS/MS analysis, a score was assigned which is a composite score based on the number of peptides matched, and the level of identity.

TABLE 1
Proteins Identified by H460-16-2 from
Western Blot of MB-468 Membranes

| Observed | | | | NCBI Accesion |
|---|---|---|---|---|
| MW | Method | Protein ID | Score | # |
| 130-150 | LC/MS/MS | KIAA0315 | 401 | gi\|2280476 |
| KD | | | | |
| 85-95 kD | LC/MS/MS | Heat shock protein | 691 | gi\|15010550 |
| | | gp96 precursor; Tumor | | |
| | | rejection antigen; grp | | |
| | | 94 | | |

TABLE 2
Proteins Identified by H460-16-2 from
Western Blot of MB-231 Membranes

| Observed | | | | NCBI Accession |
|---|---|---|---|---|
| MW | Method | Protein ID | Score | # |
| 85-95 kD | MALDI | Heat shock | (Maldi) | gi\|15010550\|gb\| |
| | | protein gp96 | | AAK74072.1 |
| | | precursor | | |
| 85-95 kD | MALDI | Tumor rejection | (Maldi) | gi\|4507677\|ref\| |
| | | antigen (gp96) 1; | | NP_003290.1 |
| | | Tumor rejection | | |
| | | antigen-1 (gp96) | | |
| 85-95 kD | LC/MS/MS | VLA-3 alpha | 429 | gi\|220141 |
| (more | | subunit actin | 212 | gi\|156763 |
| basic pI) | | Heat shock | 178 | gi\|5729877 |
| | | 70kDa protein 8 | | |
| | | isoform 1 | | |
| 130-150 | LC/MS/MS | Oxygen regulated | (Maldi) | gi\|5453832 |
| KD | | protein precursor; | | |
| | | oxygen regulated | | |
| | | protein (150kD) | | |
| 130-150 | MALDI | Protein similar to | 684 | gi\|22063141\|ref\| |
| KD | | 150 kDa oxygen- | | XP_006464.8 |
| | | regulated protein | | |
| | | precursor | | |
| | | (Orp150) | | |

All proteins were matched to human proteins. In both experiments, the predominant protein present in the 85-95 kD MW range with acidic pI was gp96 and its precursor. Alternative nomenclature for this protein includes heat shock protein (HSP) gp96, tumor rejection antigen and glucose-regulated protein, grp94. This protein was present in both experiments, and in two separate spots excised from the gel of the second experiment. In the second experiment, a portion of gel was excised in the same MW range, but with a more basic PI. In this experiment, this portion of the gel appeared to correspond with the region recognized by H460-16-2. The proteins in this portion of the gel were identified as VLA-3 alpha subunit (also called CD49c), HSP 70, and actin. The higher MW range protein was more difficult to excise reproducibly between the two experiments, likely because of the difficulty in precisely align the gel and the blot according to MW markers. In the first experiment, the 130-150 kD protein was identified as K1AA0315, alternatively known as plexin B2, which is a cell adhesion protein overexpressed in glioblastomas. In the second experiment, the proteins in this MW region identified by H460-16-2 were oxygen-regulated protein and its precursor (ORP150). These were identified in two independent gel plugs excised from the region demonstrating antibody binding. The common antigen identified from these experiments was gp96 precursor and tumor rejection antigen, gp96.

### EXAMPLE 3

### Determining Glycosylation of Antigens Bound by H460-16-2

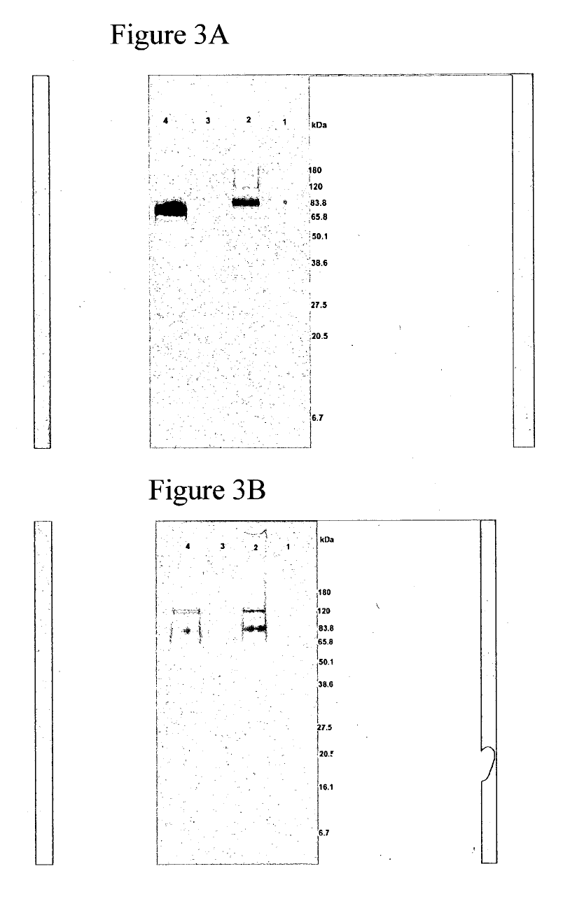

In order to determine if the antigen(s) recognized by the antibody H460-16-2 were glycoproteins, MB-468 membranes were incubated with PNGase F, Endo-o-glycosidase, and sialidase A according to manufacturer's protocol (DeglycoPro deglycosylation kit; Prozyme, San Leandro, Calif.) for 24 hr at room temperature or at 37° C. Membranes were separated by 1-D polyacrylamide gel electrophoresis as described, and then Western blotting was carried out as described with H460-16-2. The results of the Western blot are shown in FIG. 3. In MB-468 membranes that were not treated with glycosidases, H460-16-2 recognized the expected 85-95 kD band (Lane 2). In membranes treated with glycosidases at 25° C., there is a distinct shift of this band to a lower molecular weight (Lane 4). In membranes treated with glycosidases at 37° C., the binding of H460-16-2 is eliminated(Lane 3). In order to determine the completeness of deglycosylation, a similar blot was probed with the high-mannose binding lectin galanthus nivalis agglutinin (GNA). Results observed in FIG. 3, Panel B demonstrate that deglycosylation is incomplete at 25° C. and essentially complete at 37° C. These results suggest that the 85-95 kD band is a glycoprotein, and is consistent with the identification of this band as gp96/tumor rejection antigen. In addition, these results present evidence that the epitope recognized by H460-16-2 is carbohydrate-dependent.

### EXAMPLE 4

### Identification of Antigens bound by anti-gp96 Antibody

In order to determine if H460-16-2 and anti-gp96 bind the same protein, a Western blot was carried out with anti-gp96 mouse monoclonal antibodies (9G10.F8.2; Neomarkers, Fremont, Calif.). Binding of this antibody was tested against total membrane preparations from MDA-MB-231 and MDA-MB-468 cells, as well as the fraction of proteins immunoprecipitated by the antibody H460-16-2.

### Immunoprecipitation

Five hundred microliters of Protein G Dynabeads (DYNAL) were washed three times with 0.1M sodium phosphate buffer, pH 7.4. Twelve hundred and fifty micrograms of H460-16-2 was added to the washed beads in a total volume of 500 microliters. The mixture was incubated with gentle mixing for 1 hr. Unbound antibody was removed and the H460-16-2-coated beads were washed three times in 5 mL volumes of 0.1M sodium phosphate buffer, pH 7.4 containing 0.1% Tween-20. The H460-16-2-coated beads were washed twice in 5 mL of 0.2M Triethanolamine, pH 8.2 followed by an additional 5 mL. H460-16-2 was chemically crosslinked to the beads by gentle mixing in the presence of 5 mL of 0.2 M Triethanolamine, pH 8.2 containing 20 mM dimethyl pimelimidata for 30 min. The reaction was stopped by the addition of 5 mL of 50 mM Tris, pH 7.5. After 15 min. incubation, the H460-16-2 crosslinked beads were washed three times in PBS containing 0.1% Tween-20. The H460-16-2-crosslinked beads were pre-eluted by incubation with 0.1 M citrate pH 3 for 3 min. followed by three washes in 0.1 M sodium phosphate buffer, pH 7.4 containing 0.1% Tween-20.

Twenty-five hundred micrograms of total membrane protein from MB-468 cells were incubated with H460-16-2 chemically crosslinked beads in 0.1 M sodium phosphate buffer, pH 7.4 at 4° C. overnight. After overnight incubation, the immunoprecipate was washed three times with PBS. Protein was eluted twice from the beads by incubating the H460-16-2-crosslinked beads with 0.1 M citrate, pH 3 for 4 min. Eluted protein was stored at −80° C. One thirtieth of the total eluted protein was loaded per lane onto an 8% non-reducing SDS-PAGE gel. A sample of pre-stained molecular weight markers (Biorad; Mississauga, ON) was loaded in a reference lane. The sample was separated by electrophoresis at 100 V for 10 min., followed by 65 min. at 150 V. Proteins were transferred from the gel to PVDF (Millipore; Billerica, Mass.) membranes by electroblotting for 16 hr at 40 V. Quantitative transfer was assessed by noting complete transfer of the prestained markers from the gel to the membrane.

After transfer, membranes were blocked with 5% skim milk powder in Tris-buffered saline containing 0.05% Tween-20 (TBST) for 2 hr. The membrane was cut into strips and each strip was probed with 5 microgram/mL of the following antibodies diluted into 3% skim milk powder in TBST for 2 hr: H460-16-2, isotype control (Mouse anti-trinitrophenol, IgG1,k; clone 107.3 (BD Biosciences, Oakville, ON)). After washing 3 times with TBST, membranes were incubated with appropriate secondary antibody: goat anti-mouse IgG (Fc) conjugated to horse radish peroxidase (HRP) from Jackson Laboratories for 1 hr. This incubation was followed by washing 3 times with TBST, followed by incubation with the HRP substrate TMB (substrate kit from Vector Laboratories).

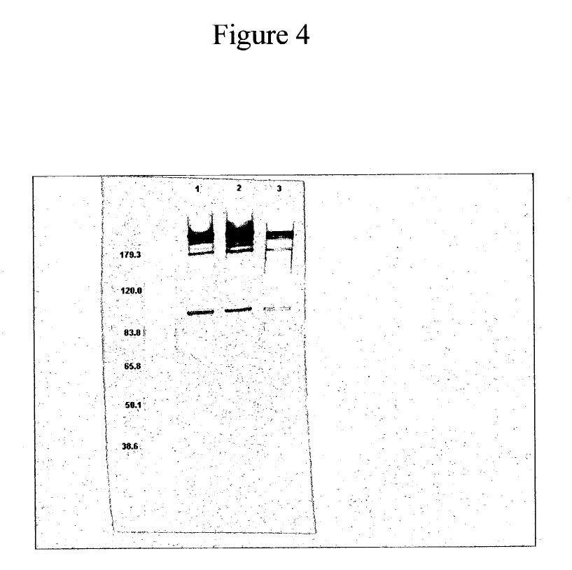

FIG. 4 shows a 1-D blot of proteins recognized by the anti-gp96 antibody 9G10.F8.2. The blot demonstrates binding to membrane proteins from MDA-MB-468 (Lane 1), MDA-MB-231 (Lane 2), and proteins immunoprecipitated by H460-16-2 (Lane 3). The position of molecular weight markers are shown in an adjacent lane. Anti-gp96 recognizes a distinct band in the molecular weight range of 90-100 kD in both MDA-MB-231 and MDA-MB-468 membranes, which corresponds to the known MW of gp96. In addition, several higher molecular weight proteins are able to bind this antibody. These proteins are potentially multivalent forms of the antigen, or are possibly gp96 tightly bound to other proteins, consistent with its role as a protein chaperone. In addition, anti-gp96 binds to the subset of proteins immunoprecipitated by H460-16-2. A band at the same molecular weight (90-100 kD) is clearly visible in this material (Lane 3), as well as a band closer to 80 kD which is not visible in the total membrane preparations. This band is identical in MW to the band identified by H460-16-2 in a similar 1-D blot. This 80 kD band, which is enriched by immunoprecipitation with H460-16-2, is recognized by the monoclonal antibody directed against gp96. Together, this evidence suggests that H460-16-2 binds a carbohydrate dependent epitope of gp96, and its smaller precursor glycoprotein.

### EXAMPLE 5

The hybridoma cell line H460-16-2 was deposited, in accordance with the Budapest Treaty, with the American Type Culture Collection, 10801 University Blvd., Manassas, Va. 20110-2209 on Sep. 4, 2002, under Accession Number PTA-4621. In accordance with 37 CFR 1.808, the depositors assure that all restrictions imposed on the availability to the public of the deposited materials will be irrevocably removed upon the granting of a patent.

### Antibody Production

H460-16-2 monoclonal antibody was produced by culturing the hybridomas in CL-1000 flasks (BD Biosciences, Oakville, ON) with collections and reseeding occurring twice/week and standard antibody purification procedures with Protein G Sepharose 4 Fast Flow (Amersham Biosciences, Baie d'Urfé, QC). It is within the scope of this invention to utilize monoclonal antibodies which are humanized, chimerized or murine antibodies. H460-16-2 was compared to a number of both positive (anti-Fas (EOS9.1, IgM, kappa, 20 mg/mL, eBioscience, San Diego, Calif.), anti-Her2/neu (IgG1, kappa, 10 mg/mL, Inter Medico, Markham, ON), anti-EGFR (C225, IgG1, kappa, 5 mg/mL, Cedarlane, Homby, ON), Cycloheximide (100 mM, Sigma, Oakville, ON), NaN₃ (0.1%, Sigma, Oakville, ON)) and negative (107.3 (anti-TNP, IgG1, kappa, 20 mg/mL, BD Biosciences, Oakville, ON), GI 55-178 (anti-TNP, IgG2a, kappa, 20 mg/mL, BD Biosciences, Oakville, ON), MPC-11 (antigenic specificity unknown, IgG2b, kappa, 20 mg/mL), J606 (anti-fructosan, IgG3, kappa, 20 mg/mL), IgG Buffer (2%)) controls in a cytotoxicity assay (Table 3). Breast cancer (MB-231, MB-468), melanoma (A2058, A375), colon cancer (HT-29), lung cancer (NCI H460, A549), ovarian cancer (OVCAR-3), prostate cancer (PC-3), and non-cancer (CCD 27sk, Hs578.Bst, Hs888 Lu) cell lines were tested (all from the ATCC, Manassas, Va.). The Live/Dead cytotoxicity assay was obtained from Molecular Probes (Eugene, Oreg.). The assays were performed according to the manufacturer's instructions with the changes outlined below. Cells were plated before the assay at the predetermined appropriate density. After 2 days, 100 microliters of purified antibody was diluted into media, and then were transferred to the cell plates and incubated in a 5% CO₂ incubator for 5 days. The plate was then emptied by inverting and blotted dry. Room temperature DPBS containing MgCl₂ and CaCl₂ was dispensed into each well from a multichannel squeeze bottle, tapped three times, emptied by inversion and then blotted dry. 50 microliters of the fluorescent Live/Dead dye diluted in DPBS containing MgCl₂ and CaCl₂ was added to each well and incubated at 37° C. in a 5% CO₂ incubator for 30 minutes. The plates were read in a Perkin-Elmer HTS7000 fluorescence plate reader and the data was analyzed in Microsoft Excel and the results were tabulated in Table 4. The data represented an average of four experiments tested in triplicate and presented qualitatively in the following fashion: 4/4 experiments greater than threshold cytotoxicity (+++), ¾ experiments greater than threshold cytotoxicity (++), 2/4 experiments greater than threshold cytotoxicity (+). Unmarked cells in Table 3 represented inconsistent or effects less than the threshold cytotoxicity. The H460-16-2 antibody produced selective cytotoxicity in A2058 melanoma cells and MB-231 breast cancer cells but did not produce cytotoxicity against the remaining cancer cells, demonstrating properties of specific cytotoxicity towards cancer cells. Importantly the isolated antibody did not produce cytotoxicity against a number of non-cancer cells such as CCD 27sk, Hs578.Bst or Hs888 Lu. The chemical cytotoxic agents induced their expected cytotoxicity while a number of other antibodies which were included for comparison also performed as expected given the limitations of biological cell assays

TABLE 3

| | Melanoma | | Breast | | Lung | | Colon | Ovary | Prostate | Normal | | |
|---|---|---|---|---|---|---|---|---|---|---|---|---|
| | A2058 | A375 | MB-231 | MB-468 | NCI-H460 | A549 | HT-29 | OVCAR-3 | PC-3 | CCD 27sk | Hs578.Bst | Hs888.Lu |
| H460-16-2 (20 μg/mL) | + | | + | | | | | | | | | |
| Positive Controls | | | | | | | | | | | | |
| Anti-Fas (20 μg/mL) | ++ | +++ | | | | | | +++ | | | | |
| Anti-Her2/neu (10 μg/mL) | | | | | | | | ++ | | | | |
| Anti-EGFR | | | | + | | | | | | | | |
| (c528, 5 μg/mL) | | | | | | | | | | | | |
| Cycloheximide (100 μM) | +++ | +++ | +++ | +++ | ++ | +++ | +++ | +++ | +++ | +++ | + | +++ |
| Negative Controls | | | | | | | | | | | | |
| IgG1 (107.3, 20 μg/mL) | | | | | | | | | | | | |
| Human IgG (10 μg/mL) | | | | | | | | | + | | | |
| IgG Buffer (2%) | | | | | | | | | | | | |

Cells were prepared for FACS by initially washing the cell monolayer with DPBS (without Ca⁺⁺ and Mg⁺⁺). Cell dissociation buffer (INVITROGEN) was then used to dislodge the cells from their cell culture plates at 37° C. After centrifugation and collection the cells were resuspended in Dulbecco's phosphate buffered saline containing MgCl₂ CaCl₂ and 25% fetal bovine serum at 4° C. (wash media) and counted, aliquoted to appropriate cell density, spun down to pellet the cells and resuspended in staining media (DPBS containing MgCl₂, CaCl₂ and 2% fetal bovine serum) at 4° C. in the presence of test antibodies (H460-16-2) or control antibodies (isotype control, anti-Her2/neu or anti-EGF-R) at 20 micrograms/mL on ice for 30 minutes. Prior to the addition of Alexa Fluor 488-conjugated secondary antibody the cells were washed once with wash media. The Alexa Fluor 488-conjugated antibody in staining media was then added for 20 minutes. The cells were then washed for the final time and resuspended in staining media containing I microgram/mL propidium iodide. Flow cytometric acquisition of the cells was assessed by running samples on a FACScan using the CellQuest software (BD Biosciences). The forward (FSC) and side scatter (SSC) of the cells were set by adjusting the voltage and amplitude gains on the FSC and SSC detectors. The detectors for the three fluorescence channels (FL1, FL2, and

TABLE 4

| Antibody | NCIH460 | A549 | Hs888Lu | HT-29 | PC-3 | OVCAR-3 | A375 | A2058 | CCD-27sk | MB-231 |
|---|---|---|---|---|---|---|---|---|---|---|
| Anti-EGFR | +(100%) | +(100%) | ++ | ++ | +(98%) | ++ | +(99%) | − | +(98%) | +++ |
| Anti-HER2/neu | − | − | − | +(32%) | − | +(43%) | +(67%) | +(31%) | − | +(22%) |
| Anti-Fas | − | − | +(48%) | +(5%) | − | − | − | − | +(9%) | − |
| H460-16-2 | +(100%) | ++ | +++ | ++ | ++ | +(33%) | +++ | +++ | ++ | +++ |

FL3) were adjusted by running cells stained with purified isotype control antibody followed by Alexa Fluor 488-conjugated secondary antibody such that cells had a uniform speak with a median fluorescent intensity of approximately 1-5 units. Live cells were acquired by gating for FSC and propidium iodide exclusion. For each sample, approximately 10,000 live cells were acquired for analysis and the results presented in Table 4.

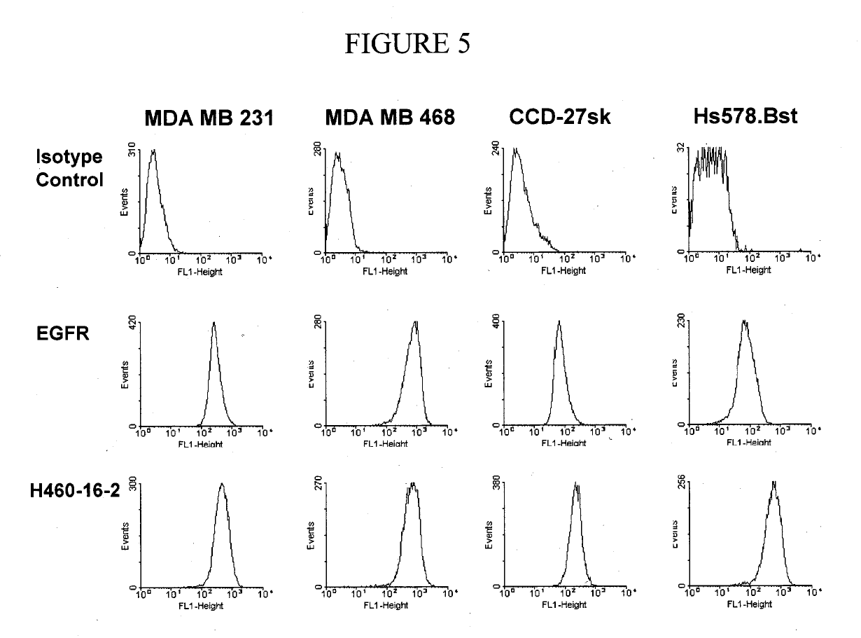

Table 4 tabulated the mean fluorescence intensity fold increase above isotype control and is presented qualitatively as: less than 5 (−); 5 to 50 (+); 50 to 100 (++); above 100 (+++) and in parenthesis, the percentage of cells stained. Representative histograms of H460-16-2 antibodies were compiled for FIG. 5 and evidence the binding characteristics, inclusive of illustrated bimodal peaks in some cases. H460-16-2 bound 100 fold above isotype control to a number of cancer cell types including melanoma and breast cancer cells; 5 to 100 fold to lung cancer cells, colon cancer cells, prostate cancer cells, and ovarian cancer cells. There was binding of H460-16-2 antibodies to non-cancer cells, however that binding did not produce cytotoxicity. This was evidence that binding was not necessarily predictive of the outcome of antibody ligation of its cognate antigen, and was a non-obvious finding. This suggested that the context of antibody ligation in different cells was determinative of cytoxicity rather than just antibody binding.

The antibodies are designed for therapeutic treatment of cancer in patients. Ideally the antibodies can be naked antibodies. They can also be conjugated to toxins, cytotoxic moieties, enzymes, radioactive compounds, and hematogenous cells. They can be used to target other molecules to the cancer. e.g. biotin conjugated enzymes. It is within the scope of the invention to provide the antibodies as murine, humanized, and chimerized.

The antibodies can be fragmented and rearranged molecularly. For example Fv fragments can be made; sFv-single chain Fv fragments; diabodies etc.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A process for mediating cytotoxicity of a human tumor cell which expresses gp96 on the cell surface comprising contacting said tumor cell with an isolated monoclonal antibody or antigen binding fragments thereof encoded by the clone deposited with the ATCC as Accession Number PTA-4621, whereby cell cytotoxicity occurs as a result of said binding.

2. The process of claim 1 wherein said isolated antibody or antigen binding fragments thereof are humanized.

3. The process of claim 1 wherein said isolated antibody or antigen binding fragments thereof are conjugated with a member selected from the group consisting of cytotoxic moieties, enzymes, radioactive compounds, and hematogenous cells.

4. The process of claim 1 wherein said isolated antibody or antigen binding fragments thereof are chimerized.

5. The process of claim I wherein said isolated antibody or antigen binding fragments thereof are murine.

6. The process of claim 1 wherein the human tumor tissue sample is obtained from a tumor originating in a tissue selected from the group consisting of colon, ovarian, lung, and breast tissue.
